(19) United States
(12) Patent Application Publication
(10) Pub. No.: US 2025/0145549 A1
(43) Pub. Date: May 8, 2025

(54) METAL VANADATES AS OXYGEN CARRIERS FOR DEHYDROGENATION

(71) Applicants: EXXONMOBIL TECHNOLOGY AND ENGINEERING COMPANY, Spring, TX (US); United States Department of Energy, Washington, DC (US)

(72) Inventors: Kun WANG, Branchburg, NJ (US); Ranjani SIRIWARDANE, MORGANTOWN, WV (US); Jarrett RILEY, MORGANTOWN, WV (US)

(21) Appl. No.: 18/930,086

(22) Filed: Oct. 29, 2024

Related U.S. Application Data
(60) Provisional application No. 63/597,080, filed on Nov. 8, 2023.

(51) Int. Cl. C07C 5/42

(52) U.S. Cl. CPC C07C 5/42

(57) ABSTRACT

Systems and methods are provided for using chemical looping oxidative dehydrogenation (CLODH) to convert alkanes, such as propane, into alkenes, such as propene. The chemical looping is performed in the presence of an oxygen carrier that includes a substantial metal vanadate phase that allows for improved conversion of alkane. Depending on the aspect, the improved conversion can correspond to an improved amount of conversion per pass during reaction, an improved selectivity for forming alkene from alkane, or a combination thereof. In some aspects, additional advantages can be provided when the metal vanadate corresponds to a Group 2 metal vanadate, such as a magnesium vanadate.

### CROSS REFERENCE TO RELATED APPLICATIONS

This Non-Provisional patent application claims priority to U.S. Provisional Patent Application No. 63/597,080, filed Nov. 8, 2023, and titled “Metal Vanadates As Oxygen Carriers For Dehydrogenation”, the entire contents of which is incorporated herein by reference.

### STATEMENT REGARDING RESEARCH

This invention was made under a CRADA, NREL Ref. No. CRD-18-00765, NETL Ref. No. AGMT-0922 among ExxonMobil Technology and Engineering Company; the National Renewable Energy Laboratory, operated for the United States Department of Energy; and the National Energy Technology Laboratory, owned and operated by the United States Department of Energy. The Government has certain rights in this invention.

### FIELD

Systems and methods are provided for performing oxidative dehydrogenation as a chemical looping process for dehydrogenation of alkanes to alkenes.

### BACKGROUND

Propene is a valuable feedstock for chemical processes, and currently demand for propene is growing. Steam cracking of naphtha, light distillate, and/or other oil by-products provides a current method for generating propene, but steam cracking is an energy intensive process with low selectivity. Thus, having improved options for commercial scale production of propene would be desirable.

One alternative to steam cracking is non-oxidative dehydrogenation of propane to propene. Equation (1) shows the non-oxidative dehydrogenation process.

C₃H₈=C₃H₆+H₂ΔH=285 KJ/mol(at 550° C.) (1)

Unfortunately, the reaction in Equation (1) is a highly endothermic process, so that an external heat source is required. The reaction is also equilibrium limited. In addition, separation of H₂ and C₃H₆ is required to get pure C₃H₆.

An alternative to non-oxidative dehydrogenation is catalytic oxidative hydrogenation. Catalytic oxidative dehydrogenation (ODH) uses oxygen and propane gas streams to convert propane into propene while forming water as a by-product instead of oxygen. Equation (2) shows the ODH reaction.

C₃H₈+O₂=C₃H₆+H₂O ΔH=4 KJ/mol(at 550° C.) (2)

As shown in Equation (2), the ODH reaction is only minimally endothermic. However, the ODH process requires a substantially pure oxygen stream. As a result, expensive air separation units are required to generate the oxygen stream for the reaction. Selectivity to obtain propene is also difficult with ODH because the simultaneous presence of propane and oxygen contributes to combustion products by the reaction of gaseous oxygen with both propane and propene. To mitigate the combustion reaction, ODH is generally conducted below 550° C., resulting in low propane conversions (<10%). In some alternative processes based on specialized catalyst supports and with addition of microwave energy, it has been reported that selectivity as high as 70% has been achieved with conversions around 20%. However, scaling of microwave-based processes to commercial scale has traditionally been a challenge. Additionally, due to the presence of propane and oxygen in the same reaction environment during the operation of ODH, additional safety precautions are typically required for a commercial scale reactor.

One option for overcoming the difficulties with conventional ODH procedures is to use chemical looping oxidative dehydrogenation (CLODH) to convert propane into propene. In CLODH, propane is converted to propene using oxygen from an oxygen carrier such as metal oxide (MO) and reduced oxygen carrier is oxidized using air in a separate reactor. This avoids mixing of oxygen from air and fuel while also avoiding expensive air separations. In CLODH, the oxygen for the reaction with propane comes solely from the oxygen carrier. In some aspects, CLODH can be performed at temperatures of 650° C. or higher because gaseous oxygen is not present.

A CLODH process operates in a different manner from an ODH process. In the ODH process, gaseous oxygen must be present with a catalyst. In conventional ODH, the reaction intermediates leading to propene production are formed on the catalyst surface using gaseous oxygen. By contrast, for a CLODH process, the oxygen for the process is provided from the solid surface where it is believed that the reaction occurs. It is noted that in CLODH, however, oxygen comes from both the surface and the bulk of the oxygen carrier. Therefore, the catalysts used in ODH will not necessarily work as oxygen carriers for CLODH.

Although CLODH can mitigate the difficulties of ODH processes, challenges remain for commercial implementation. In particular, conventional oxygen sources for CLODH processes have limited activity for performing oxidative dehydrogenation, which limits the per pass conversion at a given temperature. Increased temperatures can increase the conversion, but this also results in reduced selectivity for performing the desired conversion to propene.

Based on the above, it would be desirable to have improved systems and methods for performing CLODH processes.

U.S. Patent Application Publication 2020/0199042 describes conversion of paraffins to olefins and heavier hydrocarbons mediated by metal oxides. A variety of metal oxides for performing chemical looping are described.

EP 0403462 describes a process for the catalytic dehydrogenation of hydrocarbons using a redox catalytic system similar to the CLODH process. The examples include description of a V₂O₅-type catalyst supported on magnesium oxide as an oxygen source.

### SUMMARY

In an aspect, a method for forming alkenes, is provided. The method includes exposing an oxygen carrier containing a metal vanadate to a feedstock containing 1.0 wt % or more of a C₃-C₆ alkanes under oxidative dehydrogenation conditions to convert at least a portion of the C₃-C₆ alkanes to C₃-C₆ alkenes and to reduce at least a portion of the metal vanadate to form a reduced oxygen carrier. The oxidative dehydrogenation conditions can include a temperature of 450° C. to 700° C. The metal vanadate can include a Group 2 metal, a first row transition metal from Group 3, 4, or 6-12, or a combination thereof. Additionally, the method includes oxidizing at least a portion of the reduced metal vanadate in the presence of an oxidizing gas to form a substantially oxidized oxygen carrier, wherein the oxygen carrier comprises 30 wt % or less of the metal oxide corresponding to the Group 2 metal, the first row transition metal from Group 3, 4, or 6-12, or the combination thereof, relative to a weight of the metal vanadate in the oxygen carrier material.

### BRIEF DESCRIPTION OF THE DRAWINGS

FIG. 1 shows crystal structures for various pure phases of magnesium vanadate.

FIG. 2 shows an example of a reactor configuration for performing chemical looping oxidative dehydrogenation.

FIGS. 3A, 3B, and 3C show XRD spectra for various phases of magnesium vanadate.

FIG. 4 and FIG. 5 show results from oxidative dehydrogenation of propane in the presence of pyro-magnesium vanadate.

FIG. 6 and FIG. 7 show results from oxidative dehydrogenation of propane in the presence of ortho-magnesium vanadate.

FIG. 8 and FIG. 9 show results from oxidative dehydrogenation of propane in the presence of meta-magnesium vanadate.

FIG. 10 and FIG. 11 show results from oxidative dehydrogenation of propane in the presence of a composite phase of magnesium vanadate.

FIG. 12 and FIG. 13 show results from oxidative dehydrogenation of propane in the presence of a composite phase of magnesium vanadate in various types of reactors.

FIG. 14 shows oxygen transfer capacity for pyro-magnesium vanadate under cyclic conditions.

FIG. 15 shows oxygen transfer capacity for various magnesium vanadate phases.

FIG. 16 and FIG. 17 show results for oxidative dehydrogenation of propane in the presence of pyro-magnesium vanadate with various amounts of alumina diluent in the oxygen carrier particles at various temperatures.

FIG. 18 and FIG. 19 show results for oxidative dehydrogenation of propane in the presence of pyro-magnesium vanadate with various diluents in the oxygen carrier particles at various temperatures.

FIG. 20 shows oxygen transfer capacity for oxygen carriers containing pyro-magnesium vanadate and various diluents.

FIG. 21 and FIG. 22 shows results for oxidative dehydrogenation of propane in the presence of various metal vanadates.

FIG. 23 and FIG. 24 shows results for oxidative dehydrogenation of propane in the presence of various metal vanadates.

FIG. 25 shows results for oxidative dehydrogenation of propane in the presence of calcium vanadate under various conditions.

FIG. 26 shows results for oxidative dehydrogenation of propane in the presence of calcium vanadate under various conditions.

FIG. 27 shows an XRD spectrum for a comparative oxygen carrier.

FIG. 28 shows surface areas for oxygen carriers containing magnesium vanadate and various diluents.

### DETAILED DESCRIPTION

All numerical values within the detailed description and the claims herein are modified by “about” or “approximately” the indicated value, and take into account experimental error and variations that would be expected by a person having ordinary skill in the art.

### Overview

In various aspects, systems and methods are provided for using chemical looping oxidative dehydrogenation (CLODH) to convert propane into propene. The chemical looping is performed in the presence of an oxygen carrier that includes a substantial metal vanadate phase that allow for improved conversion of propane into propene. Depending on the aspect, the improved conversion can correspond to an improved amount of conversion per pass during reaction, an improved selectivity for forming propene from propane, or a combination thereof. In some aspects, additional advantages can be provided when the metal vanadate corresponds to a Group 2 metal vanadate, such as a magnesium vanadate.

Conventional CLODH processes can suffer from a combination of both low conversion rates for conversion of alkanes, as well as low selectivity for forming alkenes when alkanes are converted. Due in part to the need for oxygen to be removed from an oxide structure in order to perform CLODH, the conversion rate for converting alkanes in the presence of an oxide during CLODH is typically quite low. An additional difficulty is that the selectivity for forming the desired alkene product can also be low, as a portion of the alkane converted by the process can still form combustion products rather than a desired alkene product.

It has been discovered that metal vanadates can provide unexpectedly improved conversion and/or unexpectedly improved selectivity when converting alkanes to alkenes in a CLODH process. Without being bound by any particular theory, it is believed that the structure of metal vanadates can facilitate removal of an oxygen atom from the metal vanadate structure while still providing a stable oxide environment. Additionally or alternately, it is believed that at least some of the bond lengths in metal vanadate structures can be favorable for facilitating reaction of alkanes, such as propane, with oxygen provided by the metal vanadate as part of a dehydrogenation process.

It is noted that substantially pure phase metal vanadates cannot be readily formed by simply depositing vanadium oxide on a metal oxide substrate. Instead, it has been discovered that a synthesis procedure that provides more intimate contact between a metal oxide and vanadium oxide is needed in order to form substantial amounts of a metal vanadate phase (or phases).

In this discussion, references to a metal vanadate can refer to a fresh metal vanadate, a metal vanadate that is substantially fully oxidized, or a metal vanadate in a “reduced” oxidation state due to one or more oxygens having been used to perform oxidative dehydrogenation. A fresh metal vanadate corresponds to a metal vanadate that has not been reduced in the presence of an alkane, and therefore has the expected stoichiometry for the metal vanadate. A substantially fully oxidized metal vanadate is a metal vanadate either prior to any exposure to a reducing agent (such as an alkane) or after oxidation of the metal vanadate by exposure to an oxygen-containing gas (such as air, steam, CO₂, O₃, or NOₓ) under oxidation conditions for a sufficient period of time. A “reduced” metal vanadate corresponds to a metal vanadate where a portion of the oxygen in the metal vanadate has been removed via an oxidative dehydrogenation reaction, and the metal vanadate has not been exposed to oxidation conditions to return the “reduced” metal vanadate back to a substantially oxidized state.

In this discussion, the terms “metal oxide” and “metal vanadate” are used to distinguish between two different types of compounds. A “metal oxide” refers to a single metal oxide, such as magnesium oxide or vanadium oxide. A metal oxide can have an expected stoichiometry, such as MgO or V₂O₅, or the metal oxide may have a reduced stoichiometry, where a portion of the oxygen is missing after use of the metal oxide for oxidative dehydrogenation. A metal vanadate, by contrast, corresponds to an oxide phase where a mixture of metal oxides is used to form the oxide phase. The difference between a metal oxide, such as MgO or V₂O₅, and a metal vanadate such as Mg₂V₂O₇, can be readily determined using X-ray diffraction (XRD).

It is noted that a metal vanadate phase is distinct from having separate phases of metal oxide and vanadium oxide. In this discussion, when discussing the distinction between a metal vanadate phase versus separate metal oxide and vanadium oxide phases, reference will be made to a metal oxide that “corresponds” to the metal present in a metal vanadate. As an example, if the metal vanadate is a magnesium vanadate, then the corresponding metal oxides are magnesium oxide and vanadium oxide. As another example, if the metal vanadate is nickel vanadate, the corresponding metal oxides are nickel oxide and vanadium oxide. In this discussion, when characterizing the amount of a metal oxide in a composition, any metal vanadate present in the composition is not considered when determining the amount of a corresponding metal oxide. Thus, an oxygen carrier can include 50 wt % of magnesium vanadate while still containing less than 5.0 wt % of magnesium oxide, as any magnesium and oxygen atoms in the magnesium vanadate are not included as part of the amount of magnesium oxide. Similarly, any vanadium and oxygen in the magnesium vanadate is not included as part of the vanadium oxide in the composition. Again, XRD can be used to identify the amounts of metal vanadate versus metal oxide present in a composition.

In this discussion, XRD is conducted in a Panalytical X′pert Pro model X-Ray diffractometer. Fresh samples and reacted samples from the reactor tests are analyzed. For in-situ XRD experiments, about 50-70 mg of oxygen carrier are placed in an accessory hot stage (Anton Parr HTK-1200N) for controlled temperature and atmosphere experiments. The sample was heated to the reaction temperature in air and baseline XRD was obtained. After the introduction of ultra-high purity Ar at 35 ml/min, propane was injected with 5.0 ml/injection for 10 times while obtaining XRD scans. Then 100 ml of air was injected for three times. It is believed that these experiments represented reduction and oxidation conditions representative of what would be experienced in the CLODH process.

### Metal Vanadates and Synthesis Conditions

A CLODH process can be performed by exposing a feedstock containing alkanes, such as C₃-C₆ alkanes, to an oxygen carrier containing a metal vanadate. In various aspects, the oxygen carrier can contain 10 wt % or more of the metal vanadate, relative to the weight of the oxygen carrier, or 25 wt % or more of the metal vanadate, or 50 wt % or more, or 70 wt % or more, such as up to being substantially composed of metal vanadate (˜100 wt %). In such aspects, the amount of the corresponding metal oxide in the oxygen carrier can be reduced or minimized. The corresponding metal oxide for a metal vanadate refers to the metal oxide formed from the same metal used in the metal vanadate. Thus, for magnesium vanadate, the corresponding metal oxide is magnesium oxide. In some aspects, the oxygen carrier can be substantially free of the corresponding metal oxide (1.0 wt % or less relative to the weight of the metal vanadate). More generally, an oxygen carrier can contain 30 wt % or less of the corresponding metal oxide, relative to the weight of the metal vanadate, or 20 wt % or less, or 10 wt % or less, such as down to being substantially free of the corresponding metal oxide. Additionally or alternately, an oxygen carrier can contain 30 wt % or less of vanadium oxide, relative to the weight of the metal vanadate, or 20 wt % or less, or 10 wt % or less, such as down to being substantially free of vanadium oxide.

Additionally or alternately, the oxygen carrier can correspond to an oxygen carrier that includes a substantial metal vanadate phase. In this discussion, an oxygen carrier having a substantial metal vanadate phase is defined herein based on the X-ray diffraction (XRD) patter of the material. A material having a substantial metal vanadate phase is defined as a material where the relative intensity of at least one peak corresponding to the metal vanadate phase in the XRD pattern has a higher intensity than the peaks of the separate metal oxides. Thus, a material having a substantial magnesium vanadate phase is a material where at least one peak corresponding to magnesium vanadate in the XRD pattern has a larger relative intensity than any peaks corresponding to either MgO or V₂O₅. In some aspects, the primary peak corresponding to the metal vanadate in the XRD pattern can have a larger relative intensity than any of the peaks in the XRD corresponding to the separate metal oxides.

An oxygen carrier can contain a single type of metal vanadate phase (i.e., a substantially pure phase), or it can contain a composite metal vanadate phase. An example of a composite metal vanadate phase is a composite magnesium vanadate phase, where two or more of MgV₂O₆, Mg₂V₂O₇, and/or Mg₃V₂O₈ can be identified in the oxygen carrier using XRD.

In various aspects, the oxygen carrier can contain a metal vanadate phase where the metal in the metal vanadate has a +2 oxidation state when forming a metal vanadate or as a metal oxide. Examples of metals that can take have a +2 oxidation state as part of a metal vanadate or as a metal oxide include Group 2 metals (Be, Mg, Ca, Sr, Ba) and the first row transition metals other than vanadium (i.e., first row transition metal from Group 3, 4, and 6-12). The first row transition metals that can have a +2 oxidation state include Sc, Ti, Cr, Mn, Fe, Co, Ni, Cu, and Zn.

In some aspects, additional unexpected improvements in conversion activity and/or selectivity can be provided by oxygen carriers including a metal vanadate phase corresponding to magnesium vanadate, strontium vanadate, copper vanadate, molybdenum vanadate, manganese vanadate, and/or calcium vanadate. In some aspects, additional unexpected improvements in conversion activity and/or selectivity can be provided by oxygen carriers including a metal vanadate phase corresponding to a Group 2 metal vanadate, such as magnesium vanadate, calcium vanadate, and/or strontium vanadate. In some aspects, additional unexpected improvements in conversion activity and/or selectivity can be provided by oxygen carriers including a magnesium vanadate phase.

Oxygen carriers with metal vanadate phases can be formed using synthesis methods that involve intimate mixing of the starting reagents, so that metal vanadates are formed in preference to the separate metal oxides. Oxygen carriers with metal vanadate phases can be formed by either a solid synthesis route or a solution-based synthesis route.

An example of a solid synthesis method that can produce a metal vanadate phase is a solid-state granulation method. The solid-state granulation method can include the following steps: 1) mixing oxide precursor powders at roughly stoichiometric ratios to achieve a desired phase; 2) grinding for a period of time to increase fineness (reduce particle size) and aid mixing; 3) granulating the powder to desired morphology and size by gradual addition of water in a rotating vessel; and 4) heat treating at a desired temperature between 600-700° C. (30° C. below lowest melting point). In some optional aspects, an additional re-processing step including regrinding, granulation and heat treating can be performed one or more times to further aid in phase purity.

For step 1), oxide precursor powders can be mixed in amounts that provide a roughly stoichiometric ratio of metal oxide and vanadium oxide for forming the desired metal vanadate phase. For example, there are three different types of magnesium vanadate phases, corresponding to MgV₂O₆ (magnesium metavanadate), Mg₂V₂O₇ (magnesium pyrovanadate), and Mg₃V₂O₈ (magnesium orthovanadate). Thus, when forming a magnesium vanadate phase, the type of magnesium vanadate phase that forms can be controlled at least in part by choosing the desired stoichiometric ratio of MgO and V₂O₅ for the initial oxide precursor powders that are mixed together.

For step 2), the length of the grinding step can be sufficient to reduce particle size and facilitate mixing. In some aspects, the grinding can be performed for 4.0 hours to 40 hours, or 4.0 hours to 24 hours, or 10 hours to 40 hours, or 10 hours to 24 hours.

For step 3), the granulating step can be performed under conventional conditions for granulating of a powder by addition of water to a rotating vessel to achieve a desired size. The desired size can be controlled by the amount of water, rotating speed and time.

The granulation step prepares the mixture for the heating in step 4), so that as the granulated powder is heated to form the metal vanadates, the metal vanadates form in particles or granules of a target or desired size. In various aspects, the heat treating can be performed at a temperature of 600° C. to 700° C. Additionally or alternately, the maximum temperature during the heat treating can be selected so that the maximum temperature is 20° C. or more below the melting point of the lowest melting oxide in the initial mixture, or 30° C. or more, or 40° C. or more, such as down to 70° C. below. The temperature is selected as a balance of factors, as the temperature should be high enough to allow for formation of the metal vanadate phase while remaining low enough to reduce or minimize melting of any oxides, as that could result in formation of substantially larger particles due to agglomeration of melted material.

As an alternative to solid synthesis, a solution-based synthesis can be performed. As an example of a solution-based synthesis method, a citrate solution-based method was used to create relatively pure phase materials for some stoichiometries that could not be obtained by solid state methods. The solution method used ammonium metavanadate NH₄VO₃, group II metal nitrate precursors, and citric acid as the chelating agent. Stoichiometric amounts of vanadium and metal nitrate precursors based on the desired phase stoichiometry were dissolved sequentially in solution. The metal nitrate was dissolved first at 50° C. with vigorous stirring for 2.0 hours. NH₄VO₃ was added after the nitrate was dissolved. The solution was then heated to 90° C. with vigorous stirring (150-200 RPM) for 1.0 hours. Citric acid in a molar excess of roughly four times relative to the amount of the ammonium metavanadate was then added to the solution. The acidity and chelating effects of the citric acid promoted the complete dissolving of the metavanadate precursor. The solution was then condensed into a gel at 90° C. for 16 hours to 24 hours. The gel was then mantle heated to 170° C. to char the organic residues created by citric acid. The charring process created a friable black foam that was ground to fine powder. The powder was then heat treated at 600° C. for 2.0 hours with a 2.0° C./min ramp up and down from the dwell point. The resulting powder was ground once more, and the heat treatment process repeated. This was done to ensure that substantially all residual char was removed. The final powder was then granulated in the same manner as the solid-state method to form spherical particles of a desired size (53-600 microns) for flow reactor studies.

It is noted that multiple types of variations can also be used in the solution synthesis method. For example, metal acetates can be used in place of and/or in addition to metal nitrates. Other chelating agents can also be used, such as polyvinyl acetate. The forming of the gel can be performed at temperatures ranging from 70° C. to 95° C. When polyvinyl acetate is used as the chelating agent, the heating to 170° C. to char organic residues can be omitted, and instead the heat treatment can be performed directly on the gel at a temperature of 400° C. to 650° C.

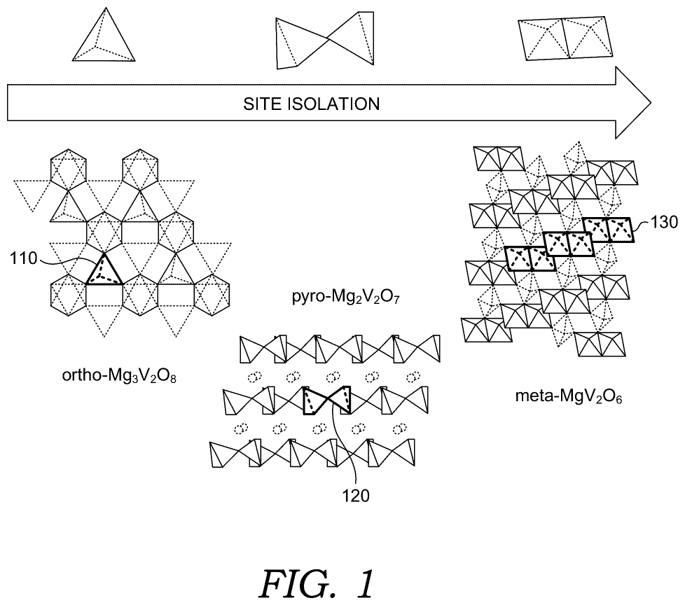

There are three types of pure vanadate phases that can be formed when combining a +2 valence metal with vanadium oxide: the ortho-phase (M3V₂O₈), the pyro-phase (M2V₂O₇), and the meta-phase (MV₂O₆). FIG. 1 illustrates examples of the structures for the ortho-, pyro-, and meta-phases in the form of magnesium vanadates. As shown in FIG. 1, the crystalline structure of each phase is different. FIG. 1 also indicates sites 110, 120, and 130, in the ortho-, pyro-, and meta-phases respectively, that roughly correspond to examples of the repeat units present within each type of phase.

The different crystal phases (ortho-, pyro-, and meta-) result in different bonding configurations and bonding distances for the various metal atoms in the metal vanadate phases. For example, for magnesium vanadates, the ortho-phase (Mg₃V₂O₈) has Mg ions in octahedral sites and V ions in tetrahedral sites. An example of a tetrahedral site for a V ion is shown in FIG. 1 as site 110. The oxygens in the ortho-phase are shared between the structures with V-O bonds having more covalent character. By contrast, for the pyro-phase (Mg₂V₂O₇), the structure consists of rows of V₂O₇ groups with long V-O bridges within these groups. Site 120 in FIG. 1 corresponds to an example of a V₂O₇ group. Longer V-O bonds in terminal V₂O₇ groups are shared by two Mg ions while shorter ones are shared with only one Mg ion. Due to this structure, the V-O bonds in the pyro-phase have less covalent character than the V-O bonds in the ortho-phase. The meta-phase (MgV₂O₆) differs from both the ortho-phase and pyro-phase, with the meta-phase having highly distorted VO₆ octahedra joined by edges and connected by MgO₆ octahedra. Site 130 in FIG. 1 corresponds to an example of a VO₆ octahedral site. All of these magnesium vanadate structures are different from the structure of V₂O₅. The structure of vanadium oxide consists of VO₅ distorted square base pyramidal units linked via two oxygens.

Without being bound by any particular theory, it is believed that these different structures in the various metal vanadate phases, which result in different bond lengths and bond energies for the metal-oxygen bonds in the structures, cause the metal vanadate structures to have unexpectedly improved activity and/or selectivity for performing oxidative dehydrogenation reactions. It is believed that propane must initially be oriented on the vanadate structures for H₂ abstraction to occur with the oxygen from the vanadates to form H₂O and propene while avoiding the competing carbon combustion reaction with the oxygens. In addition, the resulting propene, which is more reactive than propane, must also be desorbed from the vanadate structure while reducing or minimizing any combustion reactions. It is believed that metal vanadates, such as magnesium vanadates, have bond distances and/or bond energies that are more suitable (as compared to vanadium oxide and/or other oxides) for activating propane for H₂ abstraction and selective conversion to propene while reducing or minimizing combustion reactions.

In some aspects, metal vanadate particles and/or thin films (such as magnesium vanadate particles and/or thin films) can be used for performing oxidative dehydrogenation in a chemical looping system. In other aspects, the metal vanadate can be combined with a material that is relatively inert with regard to oxidative dehydrogenation. This can allow the metal vanadate to be diluted with the inert material. Examples of inert materials include oxides that are substantially inert with respect to performing oxidative dehydrogenation, such as alumina or silica. It is noted that in this discussion, “inert” metal oxides are defined to be different from any of the metals in the metal vanadate that is being diluted. For example, for a particle and/or thin film including magnesium vanadate, any magnesium oxide and vanadium oxide in the particle and/or thin film are not considered as inert metal oxides, as magnesium and vanadium are the metals used in the metal vanadate in the particle and/or thin film. As another example, for a particle and/or thin film including calcium vanadate, any calcium oxide and vanadium oxide in the particle and/or thin film are not considered as inert metal oxides.

In aspects where a metal vanadate is formulated with a diluent to form particles and/or thin films including both metal vanadate and diluent, the metal vanadate and diluent can be combined in any convenient amount. In some aspects, the amount of metal vanadate can correspond to 20 wt % to 95 wt % of the particles and/or thin film, or 20 wt % to 80 wt %, or 20 wt % to 60 wt %, or 40 wt % to 95 wt %, or 40 wt % to 80 wt %. Additionally or alternately, the amount of diluent in the particles can correspond to 5.0 wt % to 80 wt % of the particles and/or thin film, or 20 wt % to 80 wt %, or 40 wt % to 80 wt %, or 5.0 wt % to 60 wt %, or 20 wt % to 60 wt %. For characterization of a thin film, the weight of metal vanadate and weight of diluent can be determined on a localized basis, so that per square centimeter, the thin film contains 20 wt % to 80 wt % of the metal vanadate, relative to the weight of the thin film per square centimeter.

### Chemical Looping Oxidative Dehydrogenation Processes

In various aspects, one or more metal vanadates can be used as the oxygen source for performing oxidative dehydrogenation in a chemical looping environment. In some aspects, the chemical looping oxidative dehydrogenation can be performed on propane to form a product stream containing propene. In some aspects, the chemical looping oxidative dehydrogenation can be performed on C₃-C₆ alkanes to form corresponding C₃-C₆ alkenes. More generally, the chemical looping oxidative dehydrogenation can be performed on C₂-C₂₀ alkanes to form corresponding C₂-C₂₀ alkenes. Optionally, the product mixture can be substantially free of H₂, such as having an H₂ content of 500 vppm or less, or 100 vppm, such as down to 1.0 vppm or possibly still lower.

The chemical looping oxidative dehydrogenation may be performed via a cyclic process based on a circulating fluidized bed process and system, or a switched (between oxidizing gas and the hydrocarbon feedstock) feed fluidized bed system, or switched feed fixed bed system, in which oxidizing gas (such as air) and the hydrocarbon feedstock are alternated. Hydrocarbon feed can be introduced via pulse feed mode or continuous mode. Fine droplets or vapor of the hydrocarbon feedstock and oxidizing gas can be exposed to the metal vanadate bed to perform the chemical looping oxidative dehydrogenation.

In some aspects, a process can be operated in a cyclic mode without moving the solids, thus by cycling through an alkane conversion process and an oxidation process (e.g., in a reverse-flow reactor). It is noted that coating a metal vanadate on a monolith corresponds to a thin film. In other aspects, the process can be operated in a continuous fashion by moving the solid oxygen carrier or metal vanadate through an alkane conversion unit and a reduced oxygen carrier or metal vanadate oxidation unit (e.g., moving solid beds, fluidized beds). In still other aspects, the oxygen carrier based on the metal vanadate can be shaped into one or more membrane reactor(s), planar or tubular, providing continuous operations with alkane conversion and metal vanadate oxidation, which can occur on separate sides of the membrane.

In some aspects, a process cycle can include contacting a metal vanadate with an alkane, such as propane. The contacting of the metal vanadate with the alkane can result in formation of a reduced metal vanadate and a product including at least a portion of the corresponding alkene (e.g., propene formed from a propane feed). The reduced metal vanadate can then be oxidized using an oxidizing agent, such as air or another oxygen-containing gas such as steam, CO₂, O₃, or NOₓ, to form a substantially oxidized metal vanadate. During such oxidation, the reduced metal vanadate can be exposed to a partial pressure of 5.0 kPa to 150 kPa of the oxidizing agent, or 5.0 kPa to 100 kPa. The substantially oxidized metal vanadate can then be used for the next process cycle of oxidative dehydrogenation and metal vanadate oxidation.

The feedstock containing the propane and/or other alkanes can be provided in a fluid state. When the feedstock is in the gas phase, the feedstock can be exposed to the metal vanadate at a partial pressure of feedstock of 0.1 MPa to 14.0 MPa, or 0.1 MPa to 7.0 MPa, or 0.1 MPa to 1.4 Mpa. It is noted that these partial pressures can correspond to the total pressure in the reaction environment if no other gases are introduced with the feedstock.

In some aspects, contacting the metal vanadate includes cyclically exposing a fixed bed containing the metal vanadate to the alkane-containing feedstock, and then to the oxidizing gas for oxidation of the reduced metal vanadate particles and/or thin film and/or other form for the metal vanadate. Additionally or alternately, contacting the metal vanadate with the feedstock and the oxidizing gas can be performed in a fluidized bed environment. In some aspects, the gas flows delivered to the fluidized bed environment can be changed to allow for cyclic exposure to the feedstock and the oxidizing gas. In other aspects, the metal vanadate particles (optionally in a reduced state) can be circulated between one or more first reactors for performing oxidative dehydrogenation with oxygen from metal vanadate and one or more second reactors for oxidation of the reduced metal vanadate particles.

After forming a product containing alkenes, the product can be separated to form a first fraction containing at least a portion of the alkenes and a second fraction containing at least a portion of the alkanes that were not reacted during the oxidative dehydrogenation.

In aspects where particles containing the metal vanadate are rotated between reactors, the exposure of the metal vanadate to the alkane can be performed in one or more first vessels, while the exposure of the reduced metal vanadate to an oxidizing gas can be performed in one or more second vessels. From the perspective of gas flows, this simplifies exposure of the metal vanadate to the respective alkane and oxidizing gas streams.

In aspects where the chemical looping oxidative dehydrogenation is performed using metal vanadate that remains in a single vessel, such as in batch or semi-batch operation, various alternatives for managing gas flows are available. For example, when a fixed bed of metal vanadate is used and/or when the metal vanadate remains within a single reactor vessel during the oxidative dehydrogenation and oxidation cycle, various options are available for exposing the metal vanadate (and/or the reduced metal vanadate) to the feedstock and the oxidation gas flows. One option is to use sequential flows, with the feedstock and the oxidation gas alternately being exposed to the metal vanadate (and/or the reduced metal vanadate). In such an aspect, other flows could also be used between the feedstock and oxidation flows, such as purge flows. Another option can be to maintain a continuous flow of feedstock, but then periodically and/or intermittently introduce an oxidizing flow to allow for oxidation of the reduced oxygen carrier while also maintaining at least a portion of the feedstock flow.

In various aspects, the amount of feedstock exposed to the metal vanadate per cycle in the chemical looping oxidative dehydrogenation process can be characterized based on the relative weight of the metal vanadate and the alkanes in the feedstock. In some aspects, the weight ratio of metal vanadate to alkanes in the feedstock can be between 1000 (i.e., 1000 to 1) to 0.001 (i.e., 1 to 1000), or between 1000 to 1.0, or between 100 to 0.01, or between 100 to 1.0, or between 10 to 0.1, or between 10 to 1.0.

During the chemical looping oxidative dehydrogenation, the feedstock can be exposed to the metal vanadate at a temperature of 100° C. to 700° C., or 100° C. to 600° C., or 100° C. to 550° C., or 250° C. to 700° C., or 250° C. to 600° C., or 250° C. to 550° C., or 250° C. to 500° C., or 400° C. to 700° C., or 400° C. to 600° C. The total pressure during the chemical looping oxidative dehydrogenation can be 0.1 MPa-a to 14.0 MPa-a, or 0.1 MPa-a to 7.0 MPa-a, or 0.1 MPa-a to 3.5 MPa-a. The average residence time for the feedstock to be exposed to the metal vanadate can be from 0.1 seconds to 240 seconds, or 0.1 seconds to 120 seconds.

During an oxidation step, the oxidizing gas (such as air or another oxygen-containing gas) can be exposed to the reduced metal vanadate at a temperature of 50° C. to 1000° C., or 50° C. to 800° C., or 50° C. to 600° C., or 50° C. to 450° C., or 50° C. to 300° C., or 150° C. to 1000° C., or 150° C. to 800° C., or 150° C. to 600° C., or 150° C. to 450° C., or 150° C. to 300° C., or 500° C. to 1000° C., or 500° C. to 800° C., or 750° C. to 1000° C. In some aspects where a gas containing O₂ (such as air) is used as the oxidizing gas, the oxidizing gas can be exposed to the reduced metal vanadate at a temperature of 500° C. to 800° C. In some aspects where the oxidizing gas contains an oxidant other than O₂, such as water, NOₓ, or CO₂, the oxidizing gas can be exposed to the reduced metal vanadate at a temperature of 500° C. to 1000° C., or 750° C. to 1000° C. The total pressure during oxidation (of the reduced metal vanadate) can be 0.1 MPa-a to 14.0 MPa-a, or 0.1 MPa-a to 7.0 MPa-a, or 0.1 MPa-a to 3.5 MPa-a. The average residence time for the oxidizing gas to be exposed to the metal vanadate can be 1.0 seconds to 240 seconds, or 1.0 seconds to 120 seconds.

### Configuration Example

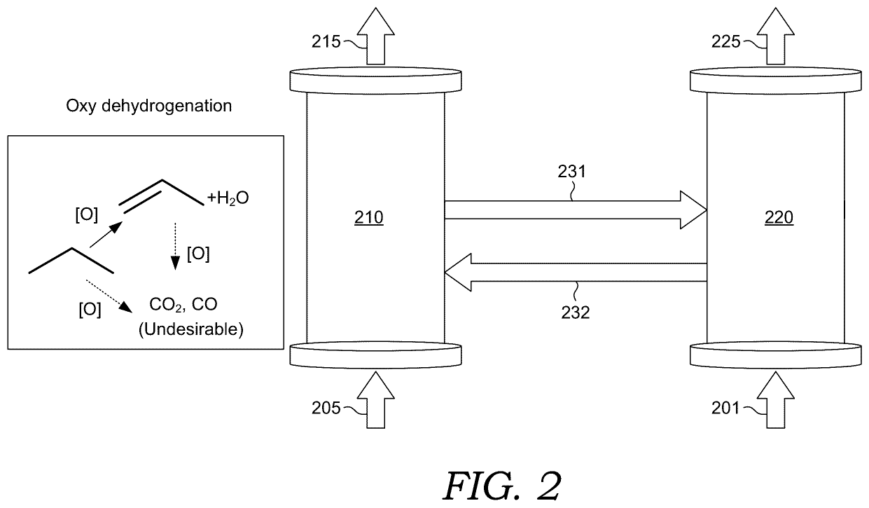

FIG. 2 shows an example of a configuration for performing oxidative dehydrogenation in a chemical looping system. In FIG. 2, reactor 210 corresponds to a reactor that contains a metal vanadate for performing oxidative dehydrogenation. In the example shown in FIG. 2, the metal vanadate is in the form of a fluidized bed and/or moving bed of metal vanadate particles. The particles can be substantially composed of one or more metal vanadates, or the particles can include one or more metal vanadates and one or more other materials, such as one or more oxides that are relatively inert under oxidative dehydrogenation conditions. An alkane-containing feed 205, such as a propane-containing feed, can be introduced into reactor 210 for exposure to the metal vanadate under oxidative dehydrogenation conditions. This produces a product effluent stream 215 containing at least some alkenes formed by conversion of alkanes. The reaction also reduces some (and up to all) of the metal vanadate particles.

In order to allow for continuous operation, at least a portion of the reduced metal vanadate particles can be passed 231 into reactor 220 for oxidation. It is noted that some substantially oxidized particles may also be included with the reduced metal particles. Reactor 220 contains a second fluidized bed/transport bed and/or moving bed of particles to allow for exposure of the particles in reactor 220 to an oxidizing gas flow 201 under oxidation (oxidizing) conditions. For example, oxidizing gas flow 201 can correspond to air, steam, CO₂, O₃, or NOₓ. The oxidizing gas can convert reduced metal vanadate particles into substantially oxidized metal vanadate particles. The substantially oxidized metal vanadate particles can then be returned 232 to the first reactor to allow for continuous operation of the oxidative dehydrogenation reaction. When air is used as the oxidizing gas for reactor 220, the oxidation also produces O₂-depleted air 225.

FIG. 2 also illustrates examples of the desired and undesired reactions in the oxidative dehydrogenation reactor 210. The desired reaction during oxidative dehydrogenation is the dehydrogenation of an alkane (such as propane) to the corresponding alkene (such as propene). In this reaction, water is preferably also formed rather than H₂. Although the metal vanadate provides oxygen in the reaction environment, performing combustion of either the initial alkane (e.g., propane) or the alkene formed by dehydrogenation (e.g., propene) is not desired. It has been discovered that using metal vanadates as the oxygen source can improve the activity for performing the dehydrogenation reaction and/or the selectivity for forming propene as opposed to other reaction products.

It is noted that the configuration in FIG. 2 provides some advantages. First, by performing the alkane dehydrogenation in a different reactor than the metal vanadate oxidation, the amount of mixing of the alkane feedstock and the oxidizing gas is reduced, minimized, or eliminated. Additionally, by using air, the need for using an air separation unit and/or another type of oxygen concentration process is eliminated, while also providing enough flow to maintain the particle bed in a fluidized state.

### Examples—Materials Preparation and Test Methods

Material preparation: Two different methods were employed for metal Vanadate preparations—a solid-state method and a solution-based method. Some metal vanadates can be prepared as pure phase metal vanadates using the solid-state method, while some others require a solution-based method to obtain a pure phase.

For the materials in these examples, unless otherwise specified, the solid-state method consisted of the following steps: Mixing oxide precursor powders at stoichiometric ratios for a desired phase; grinding for 12 hours to increase fineness and aid mixing; granulating the powder to desired morphology and size by gradual addition of water in a container with a rotor; and heat treating at a desired temperature between 600° C.-700° C. When a re-processing step was used to further improve formation of metal vanadates, the re-processing included regrinding, granulation and heat treatment at a temperature determined from melting point analysis (30° C. below lowest melting point of the precursor powders).

For the materials in these examples, unless otherwise specified, the solution-based method included the following steps: Using V₂O₅ and metal acetate (or nitrate) precursor: dissolve both precursors in water separately; combine the solutions; add poly vinyl acetate (PVA); drive off moisture and gel at 80° C.; and heat treat at 450° C. in air for 24 hrs.

Fixed bed flow reactor tests: Fixed bed flow reactor tests were conducted with AutoChem 2902II Micromeritics reactor. About 500 mg of oxygen carrier was placed in the reactor and heated up to the reaction temperature in argon. Then 5.0 vol % propane in argon was introduced to the reactor at 50 ml/min. The reaction times were limited to 5.0 min. The effluent gas concentrations were measured using a Pfeiffer GSD 301 mass spectrometer and Inficon Fusion micro-gas chromatograph.

Multi-cycle tests in Thermogravimetric Analyzer: Cyclic reduction oxidation tests with the oxygen carriers were conducted in a thermogravimetric analyzer (TA model Q50). Reductions at the reaction temperature were conducted with 5.0 vol % C₃H₈. After an argon purge, 10% O₂ balanced in Ar was introduced for oxidation.

X-ray diffraction (XRD): XRD was conducted in a Panalytical Xpert Pro model X-Ray diffractometer. Fresh samples and reacted samples from the reactor tests were analyzed. For in-situ XRD experiments, about 50-70 mg of oxygen carrier was placed in an accessory hot stage (Anton Parr HTK-1200N) for controlled temperature and atmosphere experiments. The sample was heated to the reaction temperature in air and baseline XRD was obtained. After the introduction of ultra-high purity Ar at 35 ml/min, propane was injected with 5.0 ml/injection for 10 times while obtaining XRD scans. Then 100 ml of air was injected for three times. It is believed that these experiments represented reduction and oxidation conditions representative of what would be experienced in the CLODH process.

Acidity-Basicity characterization via ammonia sorption and temperature programmed desorption (TPD): Ammonia sorption and TPD was conducted using AutoChem 2902II Micromeritics reactor. 0.2 g of oxygen carrier samples was exposed to 15 vol % NH₃/He at 100° C. during the surface loading phase and inert gas (He) was introduced at a flow rate of 15 cc/min for 30 mins at 100° C. to remove residual ammonia from the sample. The sample was cooled to 50° C. under He at the flow conditions previously listed. Then a temperature ramp was conducted at 20° C./min from 50° C. to 600° C. Desorbed NH₃ signal was measured using a TCD detector. The TPD method and instrument calibration were based on H-ZSM5 zeolite as the reference.

### Example 1—Mg Vanadates

A series of magnesium vanadates were prepared. Pure phases of meta-, ortho-, and pyro-magnesium vanadate were prepared. The pyro and ortho forms were prepared using the solution-based method. The meta form was prepared using solid state mixing method. A mixed Mg vanadate composed primarily of meta and pyro (with low levels of ortho) was also prepared using the solid state mixing method.

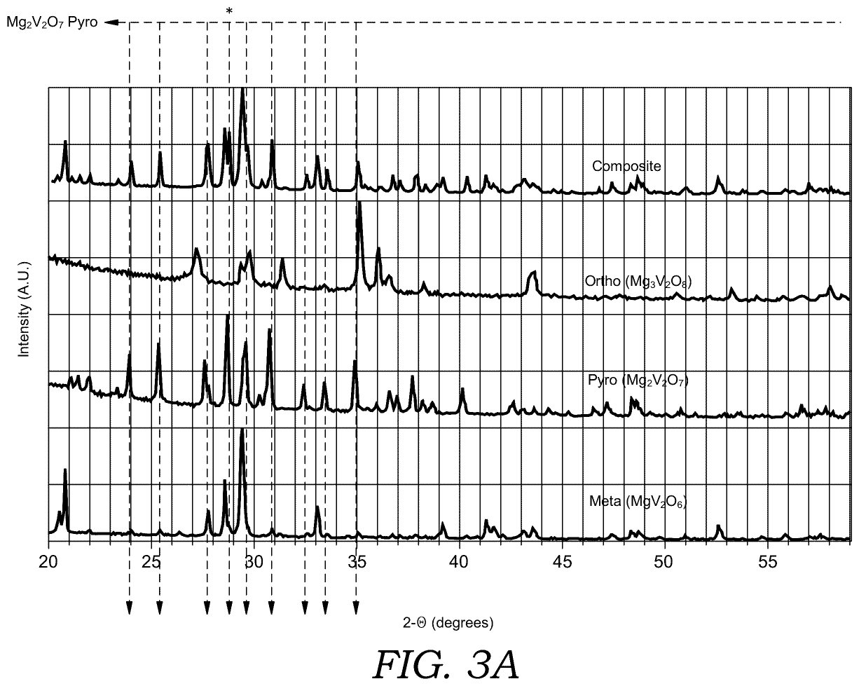

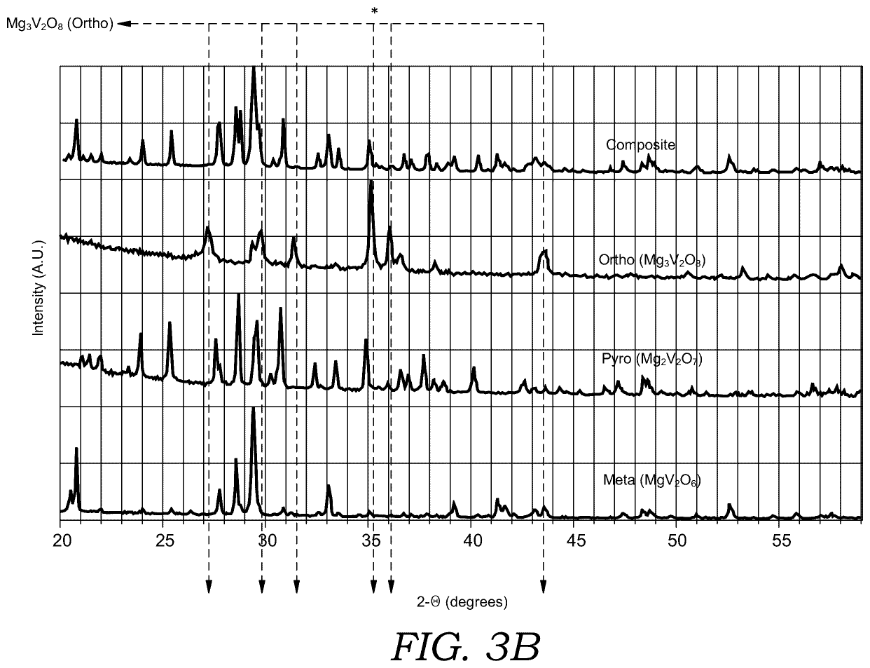

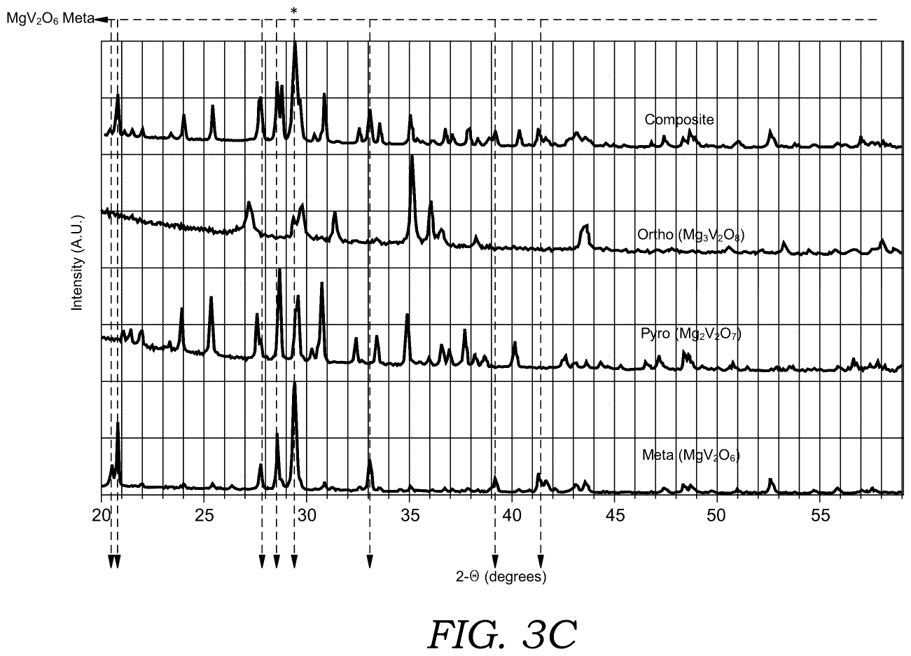

FIGS. 3A, 3B, and 3C show X-Ray diffraction data for the various magnesium vanadate materials. The XRD spectra of the three pure phase materials indicate the formation of the desired pure pyro, ortho and meta phases, respectively. In FIG. 3A, the expected peak locations for the pyro phase are also illustrated. In FIG. 3B, the expected peak locations for the ortho phase are illustrated. In FIG. 3C, the expected peak locations for the meta phase are illustrated. Based on the XRD, the composite phase material consisted of 55.4% of pyro and 42.1% of meta and 2.5% ortho phases.

Samples of the Mg vanadates shown in FIGS. 3A, 3B, and 3C were sized to a range of 53 μm to 180 μm and used in fixed bed flow reactor tests. During the fixed bed flow reactor tests with all Mg vanadates the main species observed in the effluent gas were propene, CO₂, and CO and minor amounts of H₂ and ethylene were also observed.

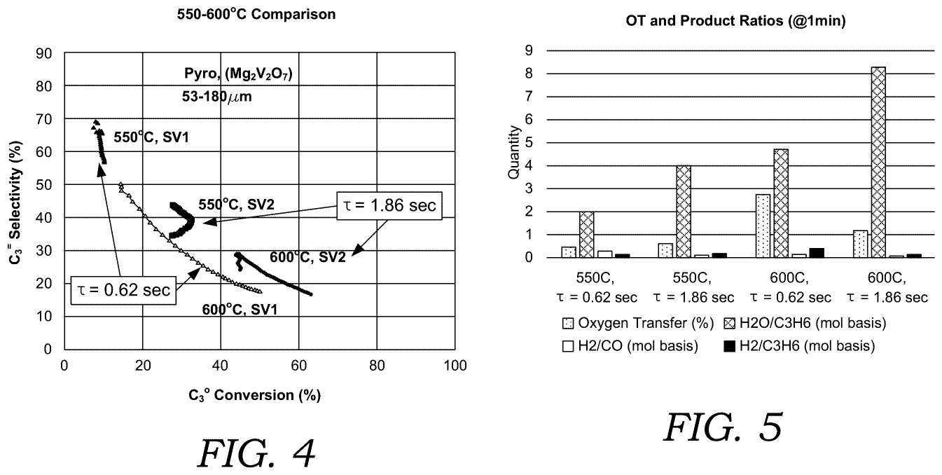

The propene selectivity as function of propane conversion with pyro Mg Vanadate (Mg₂V₂O₇) at various reaction conditions is shown in FIG. 4. Propane conversion increased with increasing temperature, but propene selectivity decreased with increasing temperature. Increase in conversion with decreasing selectivity was observed when residence time was increased. Unfortunately, it is believed that in the fixed bed continuous flow reactor tests, the product propene formed at the gas inlet location flowed through the oxygen carrier (Mg vanadate) in the reactor bed forming undesirable combustion products. Oxygen transfer capacities and effluent gas concentration ratios are shown in FIG. 5. Within roughly 1.0 minutes of the reaction a high oxygen transfer capacity of about 3% was achieved. H₂O/C₃H₆ ratios were high and H₂/C₃H₆ ratios were low (<1) indicating the propene formation is primarily via utilizing the oxygen from oxygen carrier to remove hydrogens while forming water. Additionally, the direct dehydrogenation catalytic reaction to form propene and H₂ is minimal.

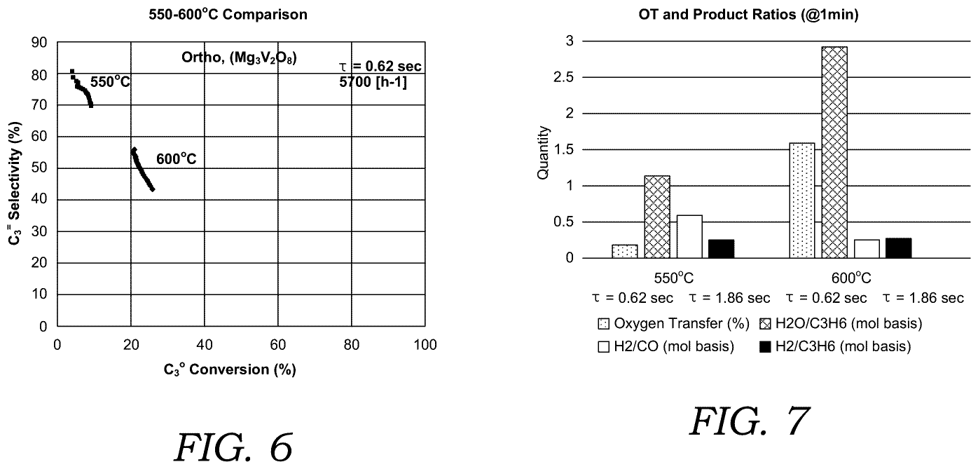

The propene selectivity as function of propane conversion with ortho Mg Vanadate (Mg₃V₂O₈) at various reaction conditions is shown in FIG. 6, while the oxygen transfer capacity of the ortho phase and the resulting product ratios from conversion are shown in FIG. 7. As shown in FIG. 6 and FIG. 7, the ortho phase showed good performance with higher selectivity and lower conversion than the pyro phase.

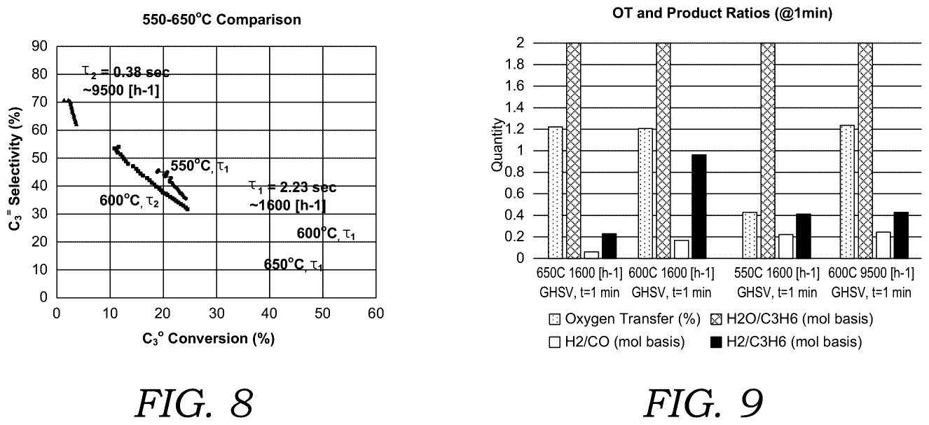

The propene selectivity as function of propane conversion with meta Mg Vanadate (MgV₂O₆) at various reaction conditions is shown in FIG. 8, while the oxygen transfer capacity of the meta phase and the resulting product ratios from conversion are shown in FIG. 9. As shown in FIG. 8 and FIG. 9, meta phase showed a reasonable performance but the conversions were lower than that with the other two vanadates. Higher conversion can be obtained at higher residence times. From the three Mg vanadate phases tested, pyro phase (FIG. 4 and FIG. 5) showed the best conversion and the ortho material (FIG. 6 and FIG. 7) showed the best selectivity. The overall performance appeared to be best with the pyro material.

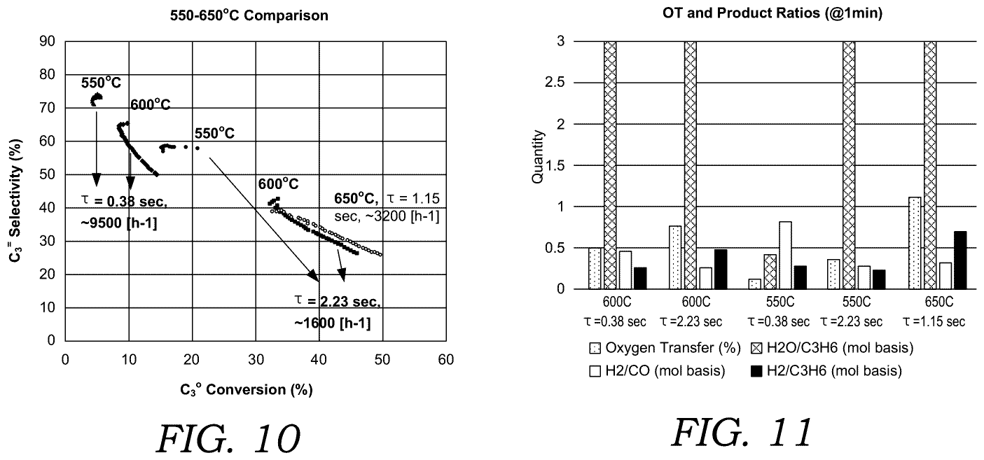

The performance of the composite phase of Mg vanadate (55.4% of pyro, 42.1% of meta, and 2.5% ortho phase) is shown in FIG. 10 and FIG. 11. This composite phase also showed high conversions in the range of 20-50% with selectivity ranging from 30-60% at 600° C. Conversion decreased and selectivity increased with increasing temperature.

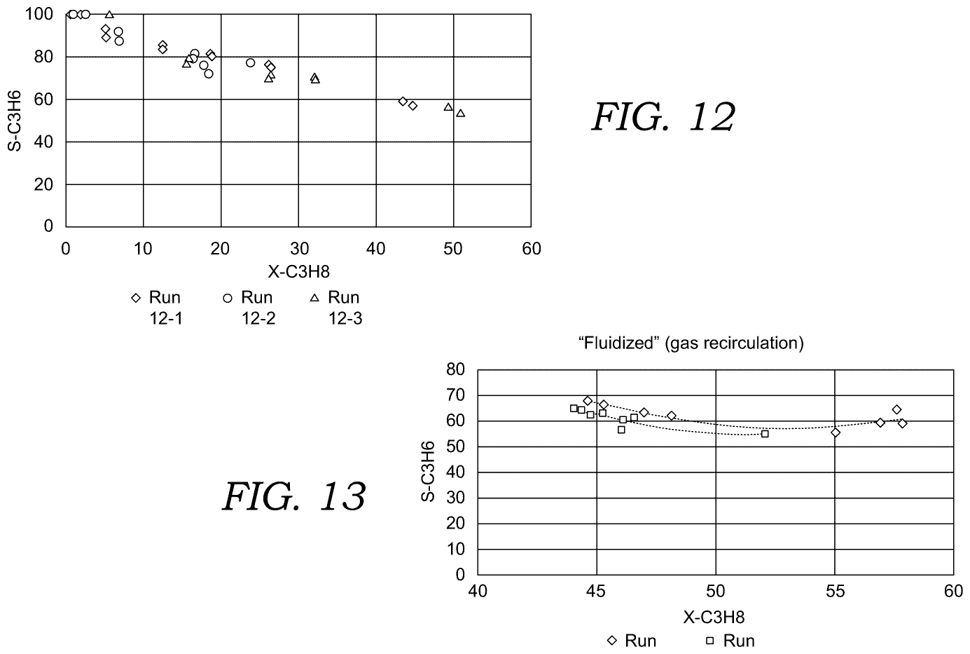

The composite phase was also used in additional testing in other types of reactors. FIG. 12 shows results from testing of the composite phase (sized to 180 μm to 600 μm) in a plug flow pulse feed reactor. The reactor had a 50 μL loop that could inject 5.0 vol % propane in He with an oxygen carrier loading of 0.12 g at a residence time of 0.1 sec. During the tests the temperature of the quartz reactor containing the oxygen carrier was heated from 400° C. to 650° C. and two pulses of 5% propane were injected at each temperature. Effluent gas composition was measured using a GC at 5.0 minute time intervals. As shown in FIG. 12, at low propane conversions, close to 100% propene selectivity was achieved with the composite Mg vanadate. As the conversion increased, selectivity decreased. For example, 80% selectivity was achieved at 25% conversion, 70% selectivity was achieved at 32% conversion, and roughly 60% selectivity was achieved at 50% propane conversion.

In another series of tests, the composite phase (sized to 180 μm to 600 μm) was tested in a small fluidized bed reactor that included an impeller to facilitate the fluidization. Although it was difficult to achieve complete fluidization of the 180 μm to 600 μm composite Mg vanadate particles in the small scale reactor due to limitations on gas flow rates, the results indicated that fluidized beds could be used for performing an oxidative dehydrogenation and metal vanadate oxidation cycle. During the test, 10 ml of propane was injected 4 times for exposure to 0.76-1.0 g of the Mg vanadate composite oxygen carrier at 600° C. After the 4^{th} propane injection, the Mg vanadate was oxidized with air and propane injections were repeated 4 times. Tests were conducted with two samples of composite Mg vanadate and the performance data are shown in FIG. 13. Data indicated high propane conversions ranging from 45%-58% with propene selectivity of 60%-70%.

### Example 2—Oxygen Transfer Capacity

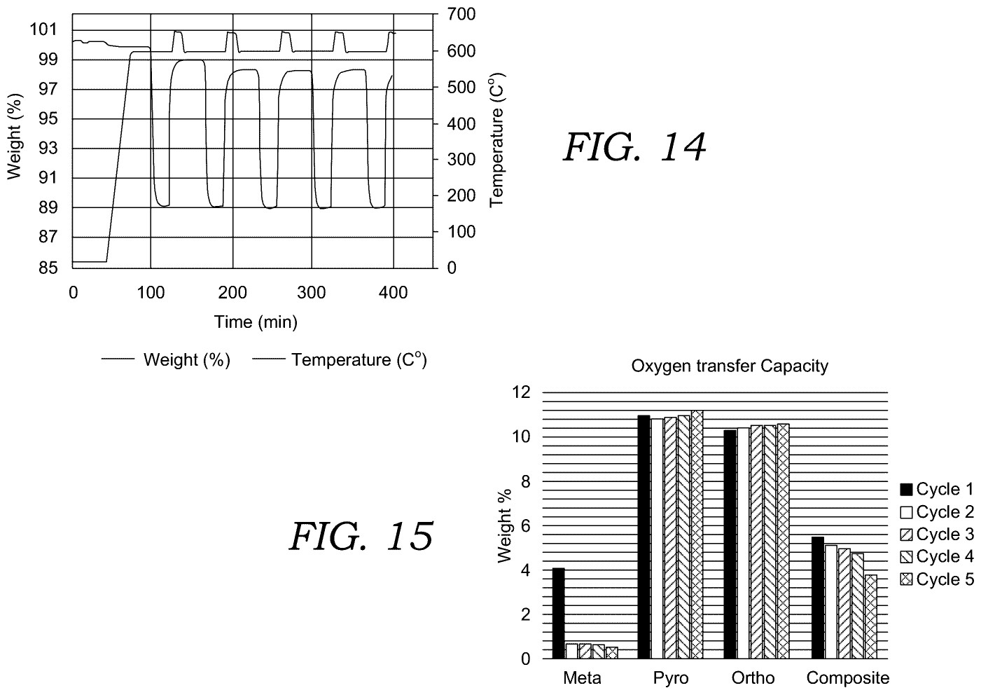

The Mg vanadates were also tested in the TGA to evaluate the oxygen transfer capacity and cyclic stability based on the TGA weight loss and weight gain data. TGA 5-cycle test data with Mg pyro vanadate are shown in FIG. 14. When 5.0 vol % C₃H₈ was introduced for ˜3 minutes at 600° C. there was a rapid weight loss. After the inert gas purge, a weight gain was observed when oxidation was conducted at 650° C. with 10 vol % O₂ for 10 minutes. As shown in FIG. 14, stable weight loss and gain profiles were observed during the 5-cycle test.

FIG. 15 shows oxygen transfer capacities based on TGA weight loss data for ortho, pyro and meta phases. Pyro phase showed high oxygen transfer capacity and cyclic stability. Ortho material also showed high oxygen transfer capacity (10 wt %) with good cyclic stability.

Additional in-situ XRD data was obtained with Mg pyro vanadate at 600° C. during oxidative dehydrogenation/oxidation cycles to monitor phase changes during the cycle. During the propane injections (oxidative dehydrogenation), transition of pyro phase to ortho phase was observed. After about 25 ml propane injections ortho phase was the most prominent phase. Slow conversion of ortho phase to a cubic phase (MgV₂O₄) was then observed with later injections (35-50 ml) and cubic phase became the most dominant phase after 50 ml propane injection. The reduced metal vanadate was then exposed to air. After exposure of 300 ml of air to the cubic phase, it was possible to reform the pyro phase. This indicates that the pyro Mg vanadate is regenerable, and therefore can be used as an oxygen carrier.

To further characterize the magnesium vanadates in both the oxidized state and the reduced state, XRD was used to determine the composition of the both pyro and ortho vanadates prior to performing oxidative dehydrogenation and after the oxidative dehydrogenation. Table 1 shows the compositional data from the characterizations.

TABLE 1
Characterization of Mg Vanadates

| | | | Lattice |
|---|---|---|---|
| Material | Primary | Secondary | Oxygen |
| Description | Phase | Phase | Removed |
| Fresh Pyro | ~100 wt % | | |
| | Mg₂V₂O₇ | | |
| Reduced | ~72.5 wt % | 15.35 wt % | 12.15 wt % |
| Pyro | MgV₂O₄ | MgO | O |
| Fresh Ortho | ~98 wt % | | |
| | Mg₃V₂O₈ | | |
| Reduced | ~70.8 wt % | 19.6 wt % | 9.6 wt % |
| Ortho | Mg_{1.3}V_{1.7}O₄ | MgO | O |

As shown in Table 1, the XRD analysis shows that both the pyro and the ortho phases can provide a substantial amount of oxygen. This is in contrast to conventional oxides for oxidative dehydrogenation, which typically can only provide less than 10% of the oxygen in the oxide. Table 2 also shows how the phase of the metal vanadate changes as the vanadate is reduced. In particular, as the metal vanadate is reduced, a portion of the metal vanadate is converted to magnesium oxide. Based on additional XRD analysis, this magnesium oxide can be substantially completely converted back to the original phase during an oxidation step.

### Example 3—Mg Vanadates with Diluents

To improve the propane conversion by promoting better gas diffusion within the granules, Mg vanadate granules were prepared by dispersing them in inert materials with high surface area. Mg vanadate particles with particle sizes less than 10 microns (ball milled) were physically mixed with high surface area inert materials in the ball mill. The granules (53-180μ) from the solid mixture were prepared by gradual addition of water to the solid mixture in a container with a rotor and heat treating at 650° C. The inert materials used included acidic θ-Al₂O₃, SiO₂, TiO₂, and basic alumina. The surface areas of these inerts ranged from 10-200 m²/g respectively. The aluminas had a surface area of roughly 200 m²/g, the SiO₂ had a surface area of roughly 100 m²/g, and the TiO₂ had a surface area of roughly 10 m²/g. The surface area of the Mg vanadate was also roughly 5.0-10 m²/g.

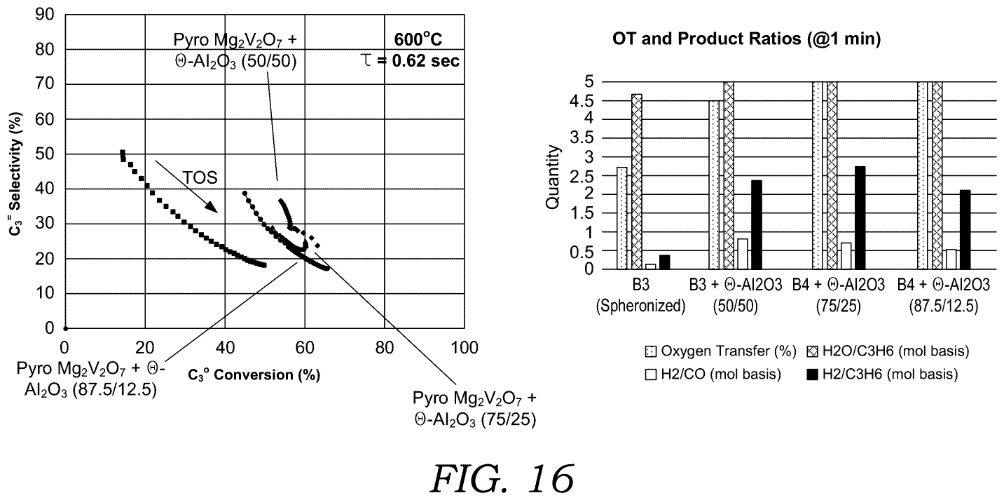

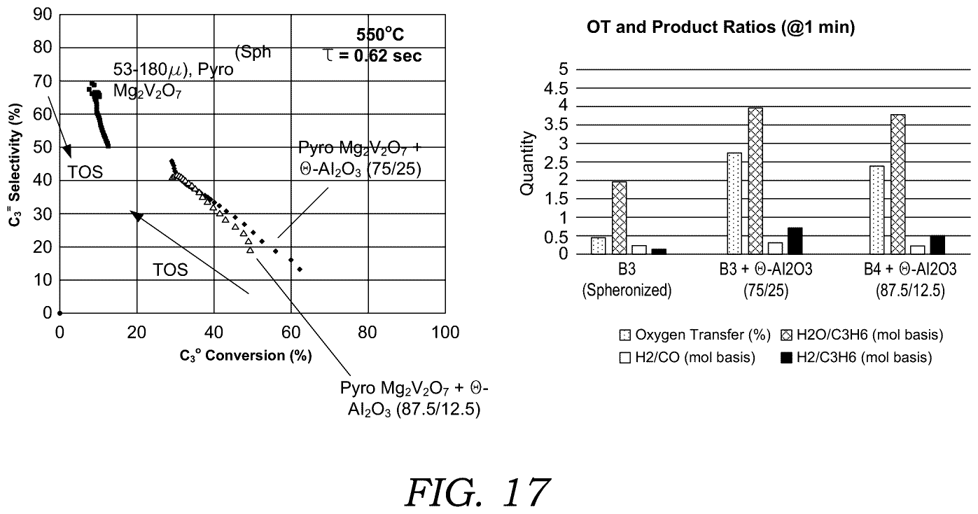

Performance data with Mg pyro vanadate diluted with 0%, 12.5%, 25% and 50% acidic θ-Al₂O₃ at 600° C. and 550° C. are shown in FIG. 16 and FIG. 17. As shown in FIG. 16 and FIG. 17, alumina dilution improved the conversion significantly. With increasing reaction time conversion decreased and selectivity increased, a trend that is opposite from that was observed with undiluted pyro phase. It is noted that for the tests at 600° C., the carbon balance was about 80-90% with the alumina diluted materials, and the H₂/propane ratio was >2 with increasing reaction time. This indicates that dehydrogenation and coking by the undesirable propane pyrolysis reaction were occurring at longer reaction times. At 550° C., the carbon balance increased and the amount of H₂ formed was also less, indicating less coking and dehydrogenation reactions at 550° C. with the alumina diluted pyro vanadate. It is further noted that temperature programmed reaction studies in propane conducted in TGA with alumina, Mg pyro Vanadate and Mg pyro vanadate/alumina indicated that reduced Mg pyro vanadate/alumina is the only phase that contributed to coking.

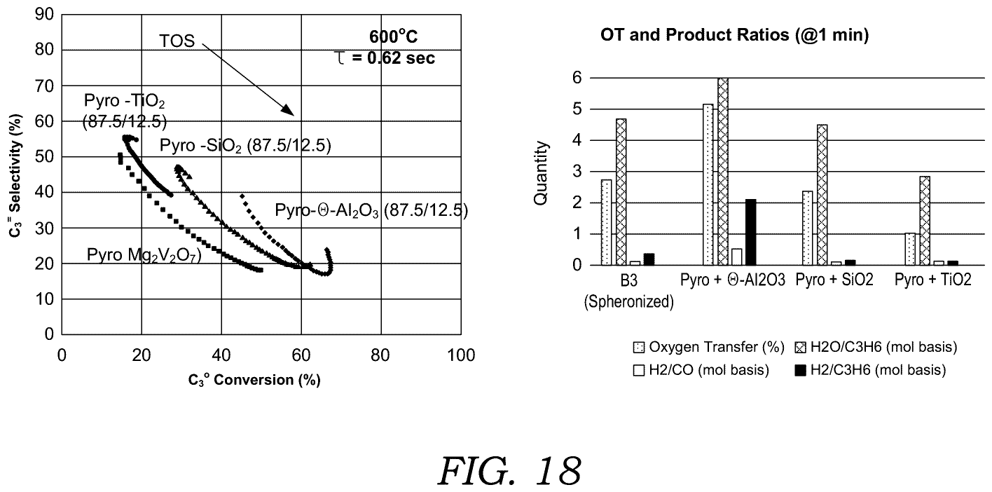

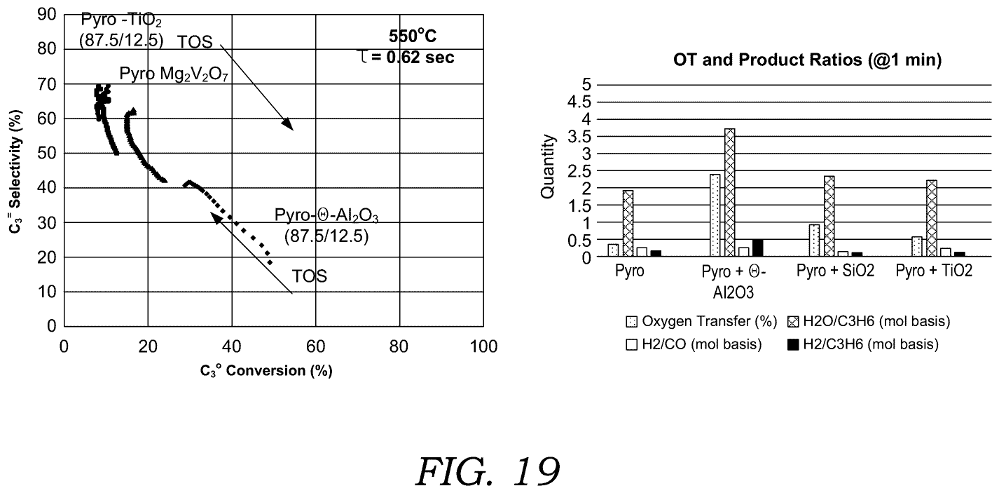

Performance data with Mg pyro vanadate diluted with TiO₂, SiO₂, and basic Al₂O₃ at 600° C. and 550° C. are shown in FIG. 18 and FIG. 19, respectively. With SiO₂ as the inert diluent the propane conversion increased while maintaining a similar selectivity. Unlike with acidic θ-Al₂O₃, with silica the carbon balance was close to 100% indicating that there is minimal carbon formation from pyrolysis with the SiO₂ containing Mg pyro vanadate. In addition, with the silica diluted oxygen carriers the selectivity decreased, and conversion increased with time. By contrast, with the θ-Al₂O₃ diluted oxygen carriers, conversion decreased and selectivity increased over time. The performance did not change significantly after diluting the Mg pyro vanadate with TiO₂ as compared to the undiluted oxygen carriers. Similar performance trends were observed at 550° C.

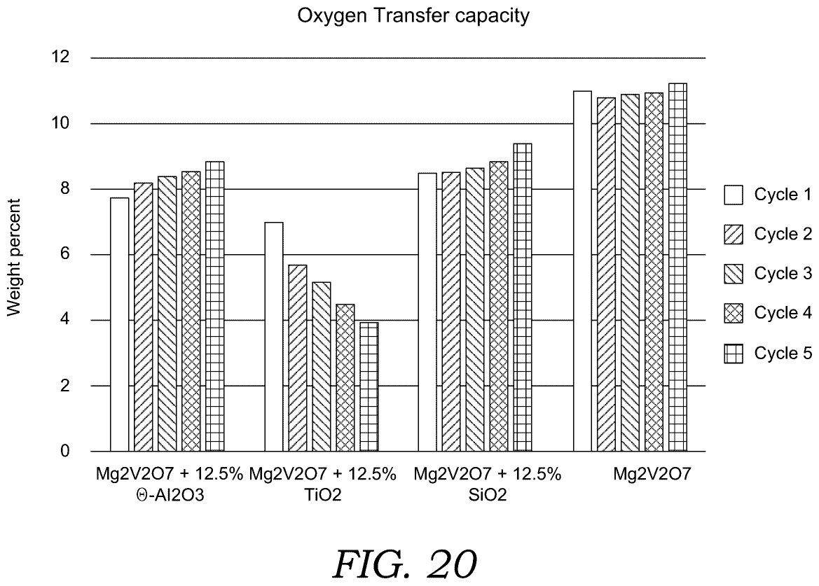

FIG. 20 shows oxygen transfer capacity during a 5-cycle TGA test with pyro Mg vanadates diluted with 12.5 wt % SiO₂, TiO₂ and alumina. The tests in FIG. 20 were performed at 600° C. Both Al₂O₃ and SiO₂ supported Mg vanadates showed good cyclic stability and high oxygen transfer capacities. TiO₂ diluted material showed poor performance.

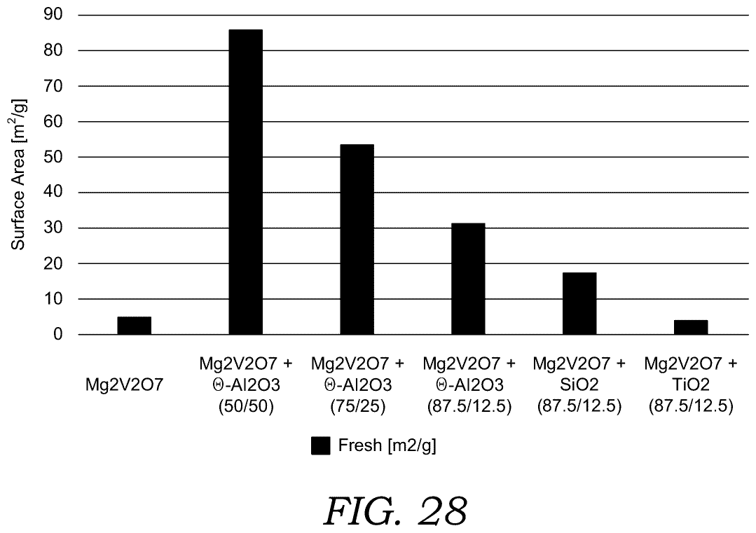

Without being bound by any particular theory, the impact of adding diluent to the metal vanadates appears to be related to the surface area of the metal vanadate versus the surface area of the diluent. The alumina (˜200 m²/g) and silica (˜100 m²/g) diluents had substantially higher surface areas than the magnesium vanadates. FIG. 28 shows the surface area for particles including the various diluents in various amounts. Adding these higher surface area diluents resulted in particles with increased surface area, and a corresponding increase in activity for performing oxidative dehydrogenation and/or a corresponding increase in selectivity. Additionally, although the diluents caused a decrease in oxygen transfer capacity of the formulated oxygen carrier, the process of diluting the oxygen carrier involves extended milling of the neat oxygen carrier with the diluent, which could create more surface area of the neat oxygen carrier. By contrast, the surface area of the TiO₂ was similar to the surface area of the undiluted magnesium vanadate. The addition of TiO₂ resulted in minimal increase in activity and/or a decrease in activity, while also substantially reducing oxygen transfer capacity. Thus, the increase in conversion activity due to addition of a diluent appears to be related to using a diluent that can increase the surface area of the oxygen carrier to 20 m²/g or more, or 40 m²/g or more, or 80 m²/g or more, such as up to 500 m²/g or possibly still higher.

Additionally or alternately, without being bound by any particular theory, the performance data can be also correlated with acidity and basicity of the Mg vanadate-inert granules as determined by temperature programmed desorption (TPD) of NH₃ data. The data from TPD of NH₃ showed high strength Bronsted acid sites with Mg vanadate/θ-Al₂O₃ granules and medium acidity with Mg vandate/SiO₂ granules. Electrophilic oxygen species (high strength acid sites) are known to activate the strong C—H bonds of propane but also contribute to total oxidation via additional C—C bond breaking. This is consistent with the high C₃H₈ conversion data observed with Mg vanadate/θ-Al₂O₃ that has electrophilic sites with multiple acid strengths. Nucleophilic sites are known to contribute to more selectivity towards C₃H₆. Therefore, it is believed that a balance of both electrophilic and nucleophilic sites can be beneficial to achieve high C₃H₆ selectivity and C₃H₈ conversion. Based on the NH₃ TPD data Mg vanadate/SiO₂ has a large number of acid cites with lower strength (Lewis sites) and high strength Bronsted acid sites were absent. Moderate acidity are known to contribute to better selectivity and Mg vanadate/SiO₂ contributed to lower conversion of C₃H₈ with higher selectivity than Mg vanadate/θ-Al₂O₃.

### Example 4—Other Metal Vanadates and Vanadium Oxide

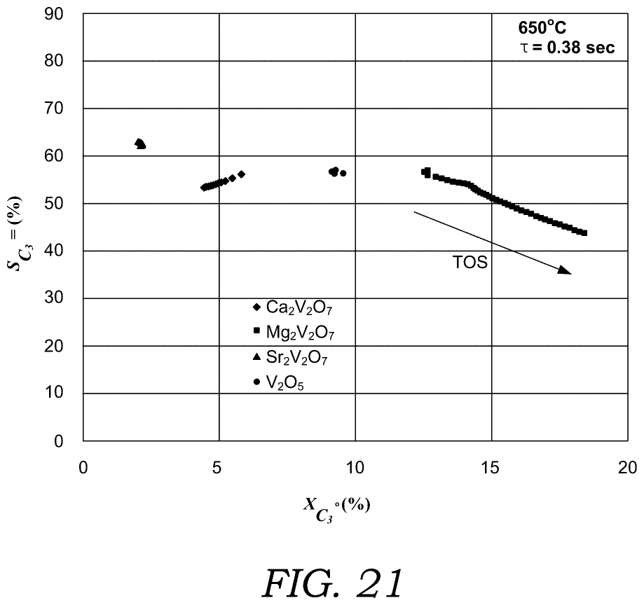

Various other metal vanadates were tested in the fixed bed flow reactor. FIG. 21 shows results from testing of Ca₂V₂O₇, Sr₂V₂O₇, Mg₂V₂O₇, and V₂O₅. As shown in FIG. 21, the magnesium vanadate provides superior activity for propane conversion relative to vanadium oxide. The calcium and strontium vanadates both have lower activity for conversion, but the strontium vanadate provides superior selectivity for formation of propene.

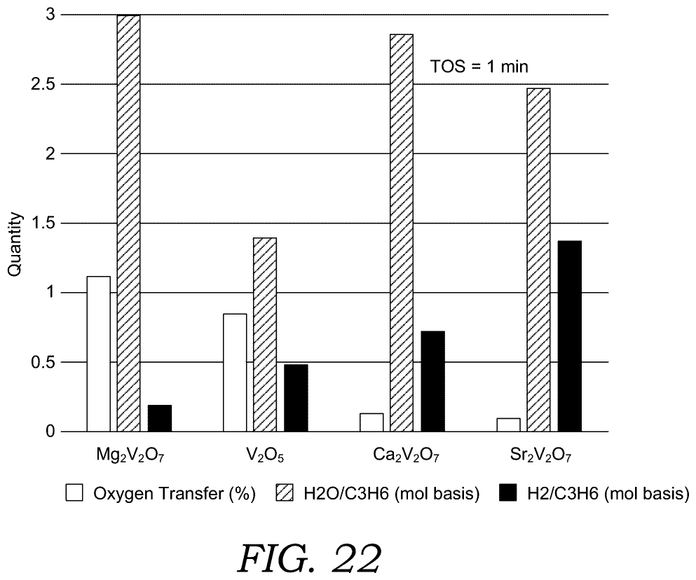

FIG. 22 provides additional details regarding the vanadates shown in FIG. 21. As shown in FIG. 22, the magnesium vanadate provides higher amounts of oxygen transfer while also reducing or minimizing the amount of H₂ produced relative to the amount of propene. This is believed to be due in part to the higher selectivity for forming water instead of H₂ from the dehydrogenation reactions facilitated by the magnesium vanadate, as compared with the vanadium oxide.

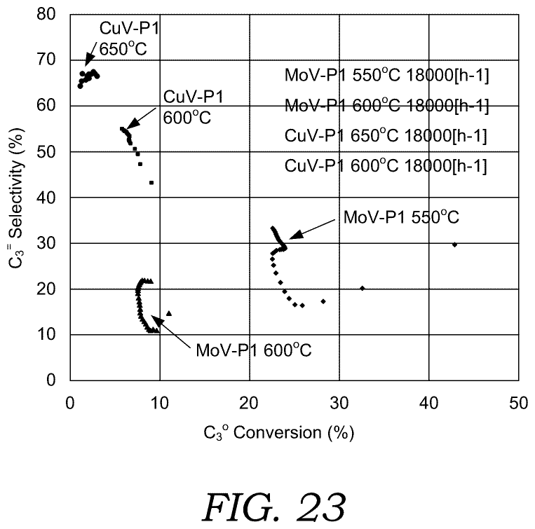

FIG. 23 shows results from testing of Cu₂V₂O₆ and Mo2V₂O₈ in the fixed bed reactor. Both copper vanadate and molybdenum vanadate are believed to perform better as the temperature is reduced. FIG. 23 shows results for the copper vanadate at 650° C. and 600° C., and molybdenum vanadate at 600° C. and 550° C. As shown in FIG. 23, both the copper and vanadate and molybdenum vanadate had higher conversion at lower temperatures. It is believed that still lower temperatures, such as 500° C. to 550° C. for copper or 450° C. to 500° C. for molybdenum, may provide further improvements in conversion.

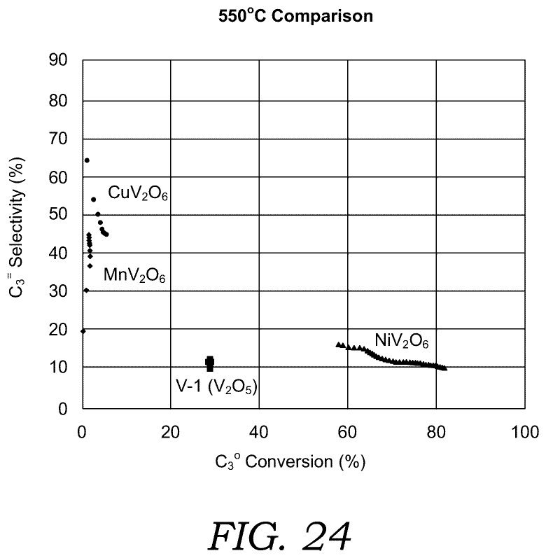

FIG. 24 shows additional results from testing of copper vanadate, nickel vanadate, manganese vanadate, and vanadium oxide at 550° C. As shown in FIG. 24, all of the metal vanadates provide superior selectivity relative to the vanadium oxide. The nickel vanadate also provides higher conversion.

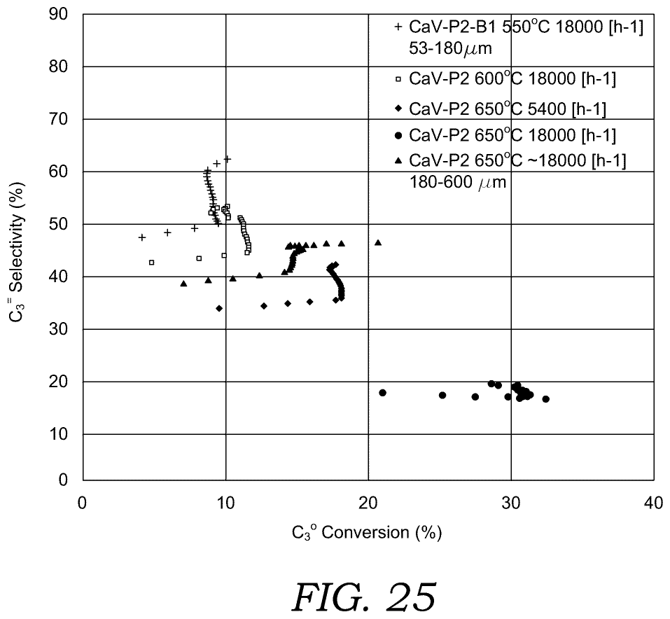

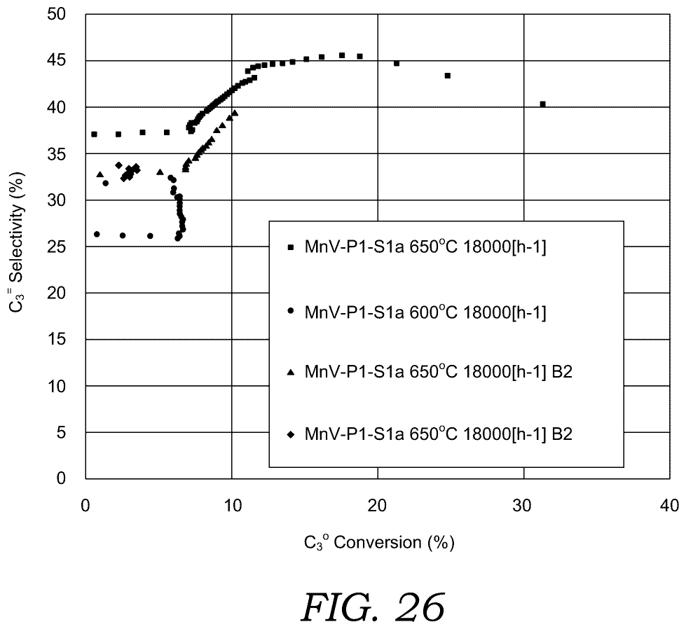

For the Ca vanadates that were tested, a composite material containing 50% CaV₂O₆/50% Ca₂V₂O₇ showed the best performance. Composite Ca vanadate showed high selectivity as shown in FIG. 25, but the propane conversion was lower than that with Mg Vanadate. Sr vanadate had the lowest conversion. FIG. 26 shows the results for Mn vanadate, which had the overall best performance of the non-Group 2 metal vanadates.

### Comparative Example 5—Synthesis of Vanadium Oxide Supported on Magnesium Oxide Particles

European patent application EP 0403462 describes performing oxidative dehydrogenation using metal oxides as the oxygen source. In an example, synthesis of a catalyst based on magnesium oxide and vanadium oxide is described. The catalyst is described in EP 04030462 as a V₂O₅ type catalyst supported on MgO.

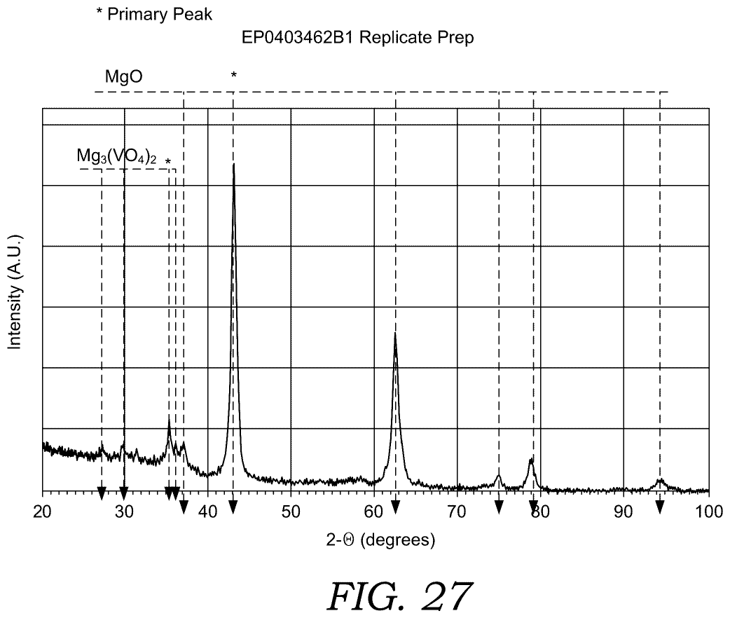

The synthesis procedure from EP 0403462 was used to form this type of catalyst. XRD was then used to characterize the resulting catalyst particles. FIG. 27 shows the XRD results. As shown in FIG. 27, the primary peaks visible in the spectrum correspond to MgO. A small amount of a magnesium vanadate is formed, but based on relative peak height, this corresponds to less than 10 wt % of the sample. This is due in part to the high excess of MgO used to create the particles. Thus, the particles formed by the method described in EP 0403462 do not correspond to magnesium vanadate particles, but instead correspond to MgO particles with an incidental amount of magnesium vanadate, and optionally some vanadium oxide supported on the particles. It is noted that the synthesis method described in EP 0403462 does not include the multiple grinding and mixing steps used to form the metal vanadates described herein. This is believed to at least partially explain why the particles formed in EP 0403462 are fundamentally difference from the metal vanadates described herein.

### ADDITIONAL EMBODIMENTS

Embodiment 1. A method for forming alkenes, comprising: exposing an oxygen carrier comprising a metal vanadate to a feedstock comprising 1.0 wt % or more of a C₃-C₆ alkanes under oxidative dehydrogenation conditions comprising a temperature of 450° C. to 700° C. to convert at least a portion of the C₃-C₆ alkanes to C₃-C₆ alkenes and to reduce at least a portion of the metal vanadate to form a reduced oxygen carrier, the metal vanadate comprising a Group 2 metal, a first row transition metal from Group 3, 4, or 6-12, or a combination thereof; and oxidizing at least a portion of the reduced metal vanadate in the presence of an oxidizing gas to form a substantially oxidized oxygen carrier, wherein the oxygen carrier comprises 30 wt % or less of the metal oxide corresponding to the Group 2 metal, the first row transition metal from Group 3, 4, or 6-12, or the combination thereof, relative to a weight of the metal vanadate in the oxygen carrier material.

Embodiment 2. The method of Embodiment 1, wherein the metal vanadate comprises a Group 2 metal, the metal vanadate optionally comprising magnesium vanadate.

Embodiment 3. The method of any of the above embodiments, wherein exposing the oxygen carrier to the feedstock comprises exposing particles of the oxygen carrier to the feedstock, or wherein exposing the oxygen carrier to the feedstock comprises exposing at least one thin film of the oxygen carrier to the feedstock, or a combination thereof.

Embodiment 4. The method of any of the above embodiments, wherein the oxygen carrier is substantially free of the metal oxide corresponding to the Group 2 metal, the first row transition metal from Group 3, 4, or 6-12, or the combination thereof.

Embodiment 5. The method of any of Embodiments 1 to 3, wherein the reduced oxygen carrier comprises 30 wt % or less of the metal oxide corresponding to the Group 2 metal, the first row transition metal from Group 3, 4, or 6-12, or the combination thereof, relative to a weight of the reduced metal vanadate in the reduced oxygen carrier.

Embodiment 6. The method of any of the above embodiments, wherein the exposing forms a product effluent comprising the C₃-C₆ alkenes, the oxidative dehydrogenation conditions comprising a conversion for C₃ alkane of 10 wt % or more, relative to a weight of C₃ alkane in the feedstock, and a selectivity for forming C₃ alkene of 20 wt % or more, relative to a weight of C₃ alkane in the feedstock.

Embodiment 7. The method of any of the above embodiments, wherein the oxidizing comprises oxidizing at least a portion of the reduced metal vanadate in the presence of an oxidizing gas at a temperature of 500° C. to 1000° C.

Embodiment 8. The method of any of the above embodiments, wherein the oxygen carrier comprises 50 wt % or more of the metal vanadate, relative to a weight of the oxygen carrier.

Embodiment 9. The method of any of the above embodiments, wherein the oxygen carrier comprises one or more metal oxides corresponding to a metal different from the Group 2 metal, the first row transition metal from Group 3, 4, or 6-12, or the combination thereof, the oxygen carrier optionally comprising a surface area of 20 m²/g or more.

Embodiment 10. The method of any of the above embodiments, wherein the feedstock comprises 10 wt % or more of C₃ alkane.

Embodiment 11. The method of any of the above embodiments, wherein the oxygen carrier comprises 30 wt % or less of vanadium oxide, relative to a weight of the metal vanadate.

Embodiment 12. The method of any of the above embodiments, wherein the oxygen carrier comprises 50 wt % or more of a substantially pure phase metal vanadate, relative to a weight of the oxygen carrier.

Embodiment 13. The method of any of the above embodiments, wherein the oxidizing gas comprises air, steam, CO₂, O₃, NOₓ, or a combination thereof.

Embodiment 14. The method of Embodiment 13, wherein the oxidizing gas comprises air, and wherein the oxidizing comprises oxidizing at least a portion of the reduced metal vanadate in the presence of an oxidizing gas at a temperature of 500° C. to 800° C., wherein.

Embodiment 15. The method of Embodiment 13, wherein the oxidizing gas comprises steam, NOₓ, or steam, and wherein the oxidizing comprises oxidizing at least a portion of the reduced metal vanadate in the presence of an oxidizing gas at a temperature of 750° C. to 1000° C.

Embodiment 16. The method of any of the above embodiments, wherein the exposing the oxygen carrier to the feedstock is performed in at least one first vessel and the oxidizing is performed in at least one second vessel, the method further comprising: passing at least a portion of the reduced oxygen carrier from the at least one first vessel to the at least one second vessel, and passing at least a portion of the substantially oxidized oxygen carrier from the at least one second vessel to the at least one first vessel.

Embodiment 17. The method of Embodiment 16, wherein the oxygen carrier comprises the at least a portion of the substantially oxidized oxygen carrier.

Additional Embodiment A. The method of any of the above embodiments, wherein the oxygen carrier comprises a substantial metal vanadate phase.

While the present invention has been described and illustrated by reference to particular embodiments, those of ordinary skill in the art will appreciate that the invention lends itself to variations not necessarily illustrated herein. For this reason, then, reference should be made solely to the appended claims for purposes of determining the true scope of the present invention.

1. A method for forming alkenes, comprising:
exposing an oxygen carrier comprising a metal vanadate to a feedstock comprising 1.0 wt % or more of a C₃-C₆ alkanes under oxidative dehydrogenation conditions comprising a temperature of 450° C. to 700° C. to convert at least a portion of the C₃-C₆ alkanes to C₃-C₆ alkenes and to reduce at least a portion of the metal vanadate to form a reduced oxygen carrier, the metal vanadate comprising a Group 2 metal, a first row transition metal from Group 3, 4, or 6-12, or a combination thereof; and
oxidizing at least a portion of the reduced metal vanadate in the presence of an oxidizing gas to form a substantially oxidized oxygen carrier,
wherein the oxygen carrier comprises 30 wt % or less of the metal oxide corresponding to the Group 2 metal, the first row transition metal from Group 3, 4, or 6-12, or the combination thereof, relative to a weight of the metal vanadate in the oxygen carrier material.

2. The method of claim 1, wherein the metal vanadate comprises a Group 2 metal.

3. The method of claim 1, wherein the metal vanadate comprises magnesium vanadate.

4. The method of claim 1, wherein exposing the oxygen carrier to the feedstock comprises exposing particles of the oxygen carrier to the feedstock, or wherein exposing the oxygen carrier to the feedstock comprises exposing at least one thin film of the oxygen carrier to the feedstock, or a combination thereof.

5. The method of claim 1, wherein the oxygen carrier is substantially free of the metal oxide corresponding to the Group 2 metal, the first row transition metal from Group 3, 4, or 6-12, or the combination thereof.

6. The method of claim 1, wherein the reduced oxygen carrier comprises 30 wt % or less of the metal oxide corresponding to the Group 2 metal, the first row transition metal from Group 3, 4, or 6-12, or the combination thereof, relative to a weight of the reduced metal vanadate in the reduced oxygen carrier.

7. The method of claim 1, wherein the exposing forms a product effluent comprising the C₃-C₆ alkenes, the oxidative dehydrogenation conditions comprising a conversion for C₃ alkane of 10 wt % or more, relative to a weight of C₃ alkane in the feedstock, and a selectivity for forming C₃ alkene of 20 wt % or more, relative to a weight of C₃ alkane in the feedstock.

8. The method of claim 1, wherein the oxidizing comprises oxidizing at least a portion of the reduced metal vanadate in the presence of an oxidizing gas at a temperature of 500° C. to 1000° C.

9. The method of claim 1, wherein the oxygen carrier comprises 50 wt % or more of the metal vanadate, relative to a weight of the oxygen carrier.

10. The method of claim 1, wherein the oxygen carrier comprises one or more metal oxides corresponding to a metal different from the Group 2 metal, the first row transition metal from Group 3, 4, or 6-12, or the combination thereof.

11. The method of claim 10, wherein the oxygen carrier comprises a surface area of 20 m²/g or more.

12. The method of claim 1, wherein the feedstock comprises 10 wt % or more of C₃ alkane.

13. The method of claim 1, wherein the oxygen carrier comprises 30 wt % or less of vanadium oxide, relative to a weight of the metal vanadate.

14. The method of claim 1, wherein the oxygen carrier comprises 50 wt % or more of a substantially pure phase metal vanadate, relative to a weight of the oxygen carrier.

15. The method of claim 1, wherein the oxidizing gas comprises air, steam, CO₂, O₃, NOₓ, or a combination thereof.

16. The method of claim 15, wherein the oxidizing gas comprises air, and wherein the oxidizing comprises oxidizing at least a portion of the reduced metal vanadate in the presence of an oxidizing gas at a temperature of 500° C. to 800° C.

17. The method of claim 15, wherein the oxidizing gas comprises steam, NOₓ, or steam, and wherein the oxidizing comprises oxidizing at least a portion of the reduced metal vanadate in the presence of an oxidizing gas at a temperature of 750° C. to 1000° C.

18. The method of claim 1, wherein the exposing the oxygen carrier to the feedstock is performed in at least one first vessel and the oxidizing is performed in at least one second vessel, the method further comprising:
passing at least a portion of the reduced oxygen carrier from the at least one first vessel to the at least one second vessel, and
passing at least a portion of the substantially oxidized oxygen carrier from the at least one second vessel to the at least one first vessel.

19. The method of claim 18, wherein the oxygen carrier comprises the at least a portion of the substantially oxidized oxygen carrier.

20. The method of claim 1, wherein the oxygen carrier comprises a substantial metal vanadate phase.
